SEQUENCE LISTING (2 sequences)

SEQ ID NO: 1 - protein, 42 aa, Homo sapiens [sequence omitted]

SEQ ID NO: 2 - protein, 12 aa, Homo sapiens
SWFEPLVEDMQR

(19) United States
(12) Patent Application Publication
(10) Pub. No.: US 2009/0142766 A1
(43) Pub. Date: Jun. 4, 2009

(54) METHODS FOR MEASURING THE METABOLISM OF CNS DERIVED BIOMOLECULES IN VIVO

(75) Inventors: David Holtzman, St. Louis, MO (US); Randall Bateman, St. Louis, MO (US); Jungsu Kim, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: Washington University in St. Louis, St. Louis, MO (US)

(21) Appl. No.: 12/267,974

(22) Filed: Nov. 10, 2008

Related U.S. Application Data
(60) Provisional application No. 60/986,756, filed on Nov. 9, 2007.

(51) Int. Cl. C12Q 1/68; C12Q 1/02

(52) U.S. Cl. 435/6; 435/29

(57) ABSTRACT

The present invention provides methods for measuring the metabolism of a central nervous system derived biomolecule implicated in a neurological and neurodegenerative disease or disorder. In particular, the method comprises measuring the in vivo metabolism of the biomolecule in the central nervous system of a subject. Also provided is a method for determining whether a therapeutic agent affects the in vivo metabolism of a central nervous system derived biomolecule.

### CROSS REFERENCE TO RELATED APPLICATIONS

This application claims the priority of U.S. provisional application No. 60/986,756, filed Nov. 9, 2007, which is hereby incorporated by reference in its entirety.

### ACKNOWLEDGEMENT OF FEDERAL RESEARCH SUPPORT

The present invention was made, at least in part, with funding from the National Institutes of Health, NIH grants R37 AG13956 and K23 AG030946. Accordingly, the United States Government may have certain rights in this invention.

### FIELD OF THE INVENTION

The invention generally relates to methods for the diagnosis and treatment of neurological and neurodegenerative diseases, disorders, and associated processes. In particular, the invention relates to a method for measuring the metabolism of central nervous system derived biomolecules in a subject in vivo.

### BACKGROUND OF INVENTION

Alzheimer's Disease (AD) is the most common cause of dementia and is an increasing public health problem. It is currently estimated to afflict 5 million people in the United States, with an expected increase to 13 million by the year 2050 (Herbert et al 2001, Alzheimer Dis. Assoc. Disord. 15(4): 169-173). AD, like other central nervous system (CNS) degenerative diseases, is characterized by disturbances in protein production, accumulation, and clearance. In AD, dysregulation in the metabolism of the protein, amyloid-beta (Aβ), is indicated by a massive buildup of this protein in the brains of those with the disease. AD leads to loss of memory, cognitive function, and ultimately independence. It takes a heavy personal and financial toll on the patient and the family. Because of the severity and increasing prevalence of this disease in the population, it is urgent that better treatments be developed.

Currently, there are some medications that modify symptoms, however, there are no disease-modifying treatments. Disease-modifying treatments will likely be most effective when given before the onset of permanent brain damage. However, by the time clinical diagnosis of AD is made, extensive neuronal loss has already occurred (Price et al. 2001, Arch. Neurol. 58(9): 1395-1402). Therefore, a way to identify those at risk of developing AD would be most helpful in preventing or delaying the onset of AD. Currently, there are no means of identifying the pathophysiologic changes that occur in AD before the onset of clinical symptoms or of effectively measuring the effects of treatments that may prevent the onset or slow the progression of the disease.

A need exists, therefore, for a sensitive, accurate, and reproducible method for measuring the in vivo metabolism of biomolecules in the CNS. In particular, a method is needed for measuring the in vivo fractional production rate and clearance rate of proteins associated with a neurodegenerative disease, e.g., the metabolism of Aβ in AD.

### BRIEF DESCRIPTION OF THE DRAWINGS

FIG. 1 depicts a schematic illustrating the processing of amyloid precursor protein (APP) into amyloid-β (Aβ) within a cell. Leucines (L), one of the possible labeling sites, are indicated in black. The amino acid sequence of Aβ (SEQ ID NO:1) is shown at the bottom, with the trypsin digest sites indicated to demonstrate the fragments that were analyzed by mass spectrometry.

FIG. 2 depicts a mass spectrometer plot showing the separation of the amyloid-β peptides. Aβ peptides were immunoprecipitated from human CSF with the central domain anti-Aβ antibody, m266, and the eluted Aβ was subjected to mass spectrometry. Mass spectral peaks are labeled with their corresponding peptide variants; Aβ₃₈, Aβ₃₉, Aβ₄₀, and Aβ₄₂.

FIG. 3 presents mass spectrometer plots illustrating the shift in molecular weight of the ¹³C-labeled Aβ₁₇₋₂₈ fragment. In panel A, unlabeled media from a human neuroglioma cell line producing Aβ in vitro was collected and immunoprecipitated. Amyloid-beta peptides were then cleaved with trypsin at sites 5, 16, and 28 (see FIG. 1) producing the two fragment envelopes shown at masses 1325 and 1336. Note the two mass envelopes of Aβ fragments Aβ₁₇₋₂₈ (1325) and Aβ₆₋₁₆ (1336) showing the statistical distribution of natural isotopes in unlabeled Aβ. Also, note there is no signal at mass of 1331, where the labeled signal would be. In panel B, media from human neuroglioma cells cultured for 24 hours in the presence of ¹³C₆-leucine was collected and Aβ was immunoprecipitated and cleaved with trypsin to produce the fragment envelopes shown at masses 1325, 1331, and 1336. Note the shift of mass (arrow) of Aβ₁₇₋₂₈ from 1325 to 1331 that demonstrates the ¹³C₆-leucine labeled Aβ fragment (Aβ*₁₇₋₂₈). Aβ₆₋₁₆ does not contain a leucine, and so is not labeled or mass shifted. A minor amount of Aβ₁₇₋₂₈ remains unlabeled.

FIG. 4 depicts a graph showing a standard curve of the labeling of Aβ in vitro. A sample of labeled cultured media was serially diluted to generate a standard curve to test the linearity and variability of the measurement technique. The Aβ was precipitated from the media, trypsin digested, and the fragments were analyzed on a Liquid Chromatography Electro-Spray Injection (LC-ESI) mass spectrometer and the tandem mass spectra ions were quantitated using custom written software. The software summed both the labeled and the unlabeled tandem ions and calculated the ratio of labeled to total Aβ. The percent labeled Aβ versus the predicted value is shown with a linear regression line. Note the good linear fit, in addition to the low deviation.

FIG. 5 depicts two graphs showing the fractional clearance rate of ApoE4 and ApoE2. Panel A illustrates the fraction of labeled ApoE to unlabelled ApoE at 1, 3, 6, 24, and 36 hours after labeling. Panel B shows the fractional clearance rate of ApoE4/4 (5.456%/h5; 95% confidence intervals: 4.394-6.518%) and ApoE2/2 (2.072%/hr; 95% confidence intervals: 1.506-2.638%/hr). The ApoE4/4 subjects were 10-12 month old mice and the ApoE2 subjects were 2-3 month old mice.

FIG. 6 depicts two graphs showing the fractional clearance rate of ApoE4 in the cortex and hippocampus. Panel A illustrates the fraction of labeled ApoE to unlabelled ApoE at 1, 6, and 24 hours after labeling in 10-12 month old apoE4/E4 mice. Panel B shows the fractional clearance rate of ApoE4 in the hippocampus (hip) (2.749%/hr; 95% confidence intervals: 1.074-4.423%) and ApoE4 in the cortex (5.456%/hr; 95% confidence intervals: 4.394-6.518%/hr).

FIG. 7 depicts two graphs showing the fractional clearance rate of Aβ in the cortex of APPswe/PS1deltaE9 double transgenic mice. Panel A illustrates fraction of labeled Aβ to unlabelled Aβ at 3, 6, and 18 hours after labeling. Panel B presents the fractional clearance rate (FCR) of soluble cortical Aβ (7.62±1.25% per hour).

### SUMMARY OF THE INVENTION

Among the various aspects of the present invention is the provision of a method for measuring the in vivo metabolism of one or more biomolecules produced in the central nervous system of a subject. The method comprises administering a labeled moiety to the subject, wherein the labeled moiety is capable of crossing the blood brain barrier and incorporating into the biomolecule(s) as the one or more biomolecules is produced in the central nervous system of the subject. The method further comprises obtaining a central nervous system sample from the subject, wherein the central nervous system sample is a central nervous system tissue. The central nervous system sample comprises a labeled biomolecule fraction in which the labeled moiety is incorporated into the one or more biomolecules, and an unlabeled biomolecule fraction in which the labeled moiety is not incorporated into the one or more biomolecules. The final step of the process comprises detecting the amount of labeled biomolecule and the amount of unlabeled biomolecule for each of the one or more biomolecules, wherein the ratio of labeled biomolecule to unlabeled biomolecule for each biomolecule is directly proportional to the metabolism of said biomolecule in the subject.

Another aspect of the invention encompasses a method for determining whether a therapeutic agent affects the metabolism of a biomolecule produced in the central nervous system of a subject. The method comprises administering a therapeutic agent and a labeled moiety to the subject, wherein the labeled moiety is capable of crossing the blood brain barrier and incorporating into the biomolecule as it is being is produced in the central nervous system of the subject. The method further comprises obtaining a biological sample from the subject, wherein the biological sample comprises a labeled biomolecule fraction in which the labeled moiety is incorporated into the biomolecule, and an unlabeled biomolecule fraction in which the labeled moiety is not incorporated into the biomolecule. The next step of the process comprises detecting the amount of labeled biomolecule and the amount of unlabeled biomolecule, wherein the ratio of labeled biomolecule to unlabeled biomolecule is directly proportional to the metabolism of the biomolecule in the subject. The final step of the process comprises comparing the metabolism of the biomolecule in the subject to a suitable control value, wherein a change from the control value indicates the therapeutic agent affects the metabolism of the biomolecule in the central nervous system of the subject.

Other aspects and features of the invention are described in more detail below.

### DETAILED DESCRIPTION OF THE INVENTION

The present invention relates to methods for determining the in vivo metabolism of central nervous system (CNS) derived biomolecules. In particular, the present invention provides a method for determining the in vivo production and clearance rates of CNS derived biomolecules that are present in CNS tissues. Consequently, in some embodiments, the methods presented allow the determination of in vivo production and clearance rates of CNS derived biomolecules in different portions of the CNS distinctly and simultaneously.

The invention also provides a method to assess whether a therapeutic agent affects the production or clearance rate of biomolecules in the CNS that are relevant to neurological or neurodegenerative diseases. Accordingly, the method may be used to determine the optimal doses and/or optimal dosing regimes of the therapeutic agent. Additionally, the method may be used to determine which subjects respond better to a particular therapeutic agent. For example, subjects with increased production of the biomolecule may respond better to one therapeutic agent, whereas subjects with decreased clearance of the biomolecule may respond better to another therapeutic agent. Alternatively, subjects with one particular genotype may respond better to a particular therapeutic agent than those with a different genotype.

### I. Methods for Monitoring the Metabolism of CNS Derived Biomolecules

The current invention provides methods for measuring the metabolism of one or more CNS derived biomolecules. By using this method, one skilled in the art may be able to study possible changes in the metabolism (e.g., production and clearance) of one or more relevant CNS derived biomolecule implicated in a particular disease state in the actual CNS tissue from which the biomolecule is derived. In addition, the invention permits the measurement of the pharmacodynamic effects of disease-modifying therapeutics in a subject.

In particular, this invention provides a method to label one or more biomolecules as the biomolecule(s) is produced in the central nervous system, to collect a CNS sample containing the labeled and unlabeled biomolecules, and to quantitate the amount of labeled and unlabeled biomolecules for each of the one or more biomolecules. These measurements may be used to calculate metabolic parameters, such as the production and clearance rates within the CNS, as well as others, for each of the one or more biomolecules of interest.

### (a) Neurodegenerative Diseases

Those of skill in the art will appreciate that the method of the invention may be used to determine the metabolism of CNS derived biomolecules implicated in several neurological and neurodegenerative diseases, disorders, or processes including, but not limited to, Alzheimer's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases (e.g. Creutzfeldt-Jakob Disease, bovine spongiform encephalopathy or Mad Cow Disease, and scrapie), Lewy Body Disease, schizophrenia, and Amyotrophic Lateral Sclerosis (ALS or Lou Gehrig's Disease). It is also envisioned that the method of the invention may be used to study the normal physiology, metabolism, and function of the CNS.

The in vivo metabolism of CNS derived biomolecules may be measured in mammalian subjects. In another embodiment, the subject may be a companion animal such as a dog or cat. In another alternative embodiment, the subject may be a livestock animal such as a cow, pig, horse, sheep or goat. In yet another alternative embodiment, the subject may be a zoo animal. In another embodiment, the subject may be a research animal such as a non-human primate or a rodent. In yet another embodiment, the subject may be a human. The subject may or may not be afflicted with, or pre-disposed to, a degenerative disease or disorder listed above.

### (b) Biomolecule

The present invention provides a method for measuring the metabolism of one or more biomolecules derived from the CNS in vivo. In general, the biomolecule is produced in a neuronal cell or a glial cell in the central nervous system. The biomolecule may be a protein, a lipid, a nucleic acid, or a carbohydrate. The possible biomolecules are only limited by the ability to label them during in vivo production and collect a sample from which their metabolism may be measured.

In a preferred embodiment, the biomolecule may be a protein or proteins produced in the CNS. For example, the protein to be measured may be, but is not limited to, amyloid-β (Aβ) and its variants, soluble amyloid precursor protein (APP), apolipoprotein E (isoforms 2, 3, or 4), apolipoprotein J (also called clusterin), Tau (another protein associated with AD), phospho Tau, glial fibrillary acidic protein, alpha-2 macroglobulin, synuclein, S100B, Myelin Basic Protein (implicated in multiple sclerosis), prions, interleukins, TDP-43, superoxide dismutase-1, huntingtin, tumor necrosis factor (TNF), heat shock protein 90 (HSP90), and combinations thereof. Additional biomolecules that may be targeted include products of, or proteins or peptides that interact with, GABAergic neurons, noradrenergic neurons, histaminergic neurons, seratonergic neurons, dopaminergic neurons, cholinergic neurons, and glutaminergic neurons. In a preferred embodiment, the protein whose in vivo metabolism is measured may be an apolipoprotein E protein. In another preferred embodiment, the protein whose in vivo metabolism is measured may be Aβ or its variants.

### (c) Labeled Moiety

Several different moieties may be used to label the biomolecule of interest. Generally speaking, the two types of labeling moieties typically utilized in the method of the invention are radioactive isotopes and non-radioactive (stable) isotopes. In a preferred embodiment, non-radioactive isotopes may be used and measured by mass spectrometry. Preferred stable isotopes include deuterium (²H), ¹³C, ¹⁵N, ^{17 or 18}O, and ^{33, 34, or 36}S, but it is recognized that a number of other stable isotope that change the mass of an atom by more or less neutrons than is seen in the prevalent native form would also be effective. A suitable label generally will change the mass of the biomolecule under study such that it can be detected in a mass spectrometer. In one embodiment, the biomolecule to be measured may be a protein, and the labeled moiety may be an amino acid comprising a non-radioactive isotope (e.g., ¹³C). In another embodiment, the biomolecule to be measured may be a nucleic acid, and the labeled moiety may be a nucleoside triphosphate comprising a non-radioactive isotope (e.g., ¹⁵N). Alternatively, a radioactive isotope may be used, and the labeled biomolecules may be measured with a scintillation counter (or via nuclear scintigraphy) rather than a mass spectrometer. One or more labeled moieties may be used simultaneously or in sequence.

In a preferred embodiment, when the method is employed to measure the metabolism of proteins, the labeled moiety typically will be an amino acid. Those of skill in the art will appreciate that several amino acids may be used to provide the label of biomolecules. Generally, the choice of amino acid is based on a variety of factors such as: (1) The amino acid generally is present in at least one residue of the protein or peptide of interest. (2) The amino acid is generally able to quickly reach the site of protein production and rapidly equilibrate across the blood-brain barrier. Leucine is a preferred amino acid to label proteins that are produced in the CNS, as demonstrated in Example 1. (3) The amino acid ideally may be an essential amino acid (not produced by the body), so that a higher percent of labeling may be achieved. Non-essential amino acids may also be used; however, measurements will likely be less accurate. (4) The amino acid label generally does not influence the metabolism of the protein of interest (e.g., very large doses of leucine may affect muscle metabolism). And (5) availability of the desired amino acid (i.e., some amino acids are much more expensive or harder to manufacture than others). In one embodiment, ¹³C₆-phenylalanine, which contains six ¹³C atoms, may be used to label a CNS derived protein. In a preferred embodiment, ¹³C₆-leucine may be used to label a CNS derived protein. In another embodiment, ¹³C₆-leucine may be used to label Aβ. In yet another embodiment, ¹³C₆-leucine may be used to label an ApoE protein.

There are numerous commercial sources of labeled amino acids, both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made with known synthetic chemical processes.

### (d) Administration of the Labeled Moiety

The labeled moiety may be administered to a subject by several methods. Suitable routes of administration include intravenously, intra-arterially, subcutaneously, intraperitoneally, intramuscularly, or orally. In a preferred embodiment, the labeled moiety may be administered by intravenous infusion. In another embodiment, the labeled moiety may be orally ingested.

The labeled moiety may be administered slowly over a period of time, as a large single dose depending upon the type of analysis chosen (e.g., steady state or bolus/chase), or slowly over a period of time after an initial bolus dose. To achieve steady-state levels of the labeled biomolecule, the labeling time generally should be of sufficient duration so that the labeled biomolecule may be reliably quantified. In one embodiment, the labeled moiety may be labeled leucine and the labeled leucine may be is administered intravenously. The labeled leucine may be administered intravenously for a period of time ranging from about one hour to about 24 hours. The rate of administration of labeled leucine may range from about 0.5 mg/kg/hr to about 5 mg/kg/hr, preferably from about 1 mg/kg/hr to about 3 mg/kg/hr, or more preferably from 1.8 mg/kg/hr to about 2.5 mg/kg/hr. In another embodiment, the labeled leucine may be administered as a bolus of between about 50 and about 500 mg/kg body weight of the subject, between about 50 and about 300 mg/kg body weight of the subject, or between about 100 and about 300 mg/kg body weight of the subject. In yet another embodiment, the labeled leucine may be administered as a bolus of about 200 mg/kg body weight of the subject. In an alternate embodiment, the labeled leucine may be administered intravenously as detailed above after an initial bolus of between about 0.5 to about 10 mg/kg, between about 1 to about 4 mg/kg, or about 2 mg/kg body weight of the subject.

Those of skill in the art will appreciate that the amount (or dose) of the labeled moiety can and will vary. Generally, the amount is dependent on (and estimated by) the following factors. (1) The type of analysis desired. For example, to achieve a steady state of about 15% labeled leucine in plasma requires about 2 mg/kg/hr over about 9 hr after an initial bolus of 2 mg/kg over 10 min. In contrast, if no steady state is required, a large bolus of labeled leucine (e.g., 1 or 5 grams of labeled leucine) may be given initially. (2) The protein under analysis. For example, if the protein is being produced rapidly, then less labeling time may be needed and less label may be needed—perhaps as little as 0.5 mg/kg over 1 hour. However, most proteins have half-lives of hours to days and, so more likely, a continuous infusion for 4, 9 or 12 hours may be used at 0.5 mg/kg to 4 mg/kg. And (3) the sensitivity of detection of the label. For example, as the sensitivity of label detection increases, the amount of label that is needed may decrease.

Those of skill in the art will appreciate that more than one label may be used in a single subject. This would allow multiple labeling of the same biomolecule and may provide information on the production or clearance of that biomolecule at different times. For example, a first label may be given to subject over an initial time period, followed by a pharmacologic agent (drug), and then a second label may be administered. In general, analysis of the samples obtained from this subject would provide a measurement of metabolism before AND after drug administration, directly measuring the pharmacodynamic effect of the drug in the same subject. Alternatively, multiple labels may be used at the same time to increase labeling of the biomolecule.

### (e) Central Nervous System (CNS) Sample

The method of the invention provides that a CNS sample be obtained from the subject such that the in vivo metabolism of the labeled biomolecule may be determined. Suitable CNS samples include, but are not limited to, tissue from the central nervous system, which comprises brain tissue and spinal cord tissue. In one embodiment of the invention, the CNS sample may be taken from brain tissue, including, but not-limited to, tissue from the forebrain (e.g., cerebral cortex, basal ganglia, hippocampus), the interbrain (e.g., thalamus, hypothalamus, subthalamus), the midbrain (e.g., tectum, tegmentum), or the hindbrain (e.g., pons, cerebellum, medulla oblongata). In an alternate embodiment, the CNS sample may be collected from spinal cord tissue. In still other embodiments, CNS samples from more than one CNS region may be taken. Accordingly, the metabolism of a biomolecule may be measured in different CNS samples, e.g., in the cortex and the hippocampus, simultaneously.

CNS samples may be obtained by known techniques. For instance, brain tissue or spinal cord tissue may be obtained via dissection or resection. Alternatively, CNS samples may be obtained using laser microdissection. The subject may or may not have to be sacrificed to obtain the sample, depending on the CNS sample desired and the subject utilized.

In general when the biomolecule under study is a protein or proteins, the invention provides that a first CNS sample may be taken from a subject prior to administration of the label to provide a baseline. After administration of the labeled amino acid, one or more samples generally will be taken from the subject. As will be appreciated by those of skill in the art, the number of samples and when they will be taken generally will depend upon a number of factors such as: the type of analysis, type of administration, the protein of interest, the rate of metabolism, the type of detection, the type of subject, etc.

In one embodiment, the biomolecule may be a protein and a CNS sample may be taken within an hour of labeling. In general, CNS samples obtained during the first 12 hours after the start of labeling may be used to determine the rate of production of the protein, and CNS samples taken during 24-36 hrs after the start of labeling may be used to determine the clearance rate of the protein. In another alternative, one sample may be taken after labeling for a period of time, such as 12 hours, to estimate the production rate, but this may be less accurate than multiple samples. In yet a further alternative, samples may be taken from an hour to days or even weeks apart depending upon the protein's production and clearance rates.

If samples at different time-points are desired, more than one subject may be used. For instance, one subject may be used for a baseline sample, another subject for a time-point of one hour post labeling, another subject for a time-point six hours post labeling, etc.

### (f) Detection

The present invention provides that detection of the amount of labeled biomolecule and the amount of unlabeled biomolecule in the CNS sample may be used to determine the ratio of labeled biomolecule to unlabeled biomolecule. Generally, the ratio of labeled to unlabeled biomolecule is directly proportional to the metabolism of the biomolecule. Suitable methods for the detection of labeled and unlabeled biomolecules can and will vary according to the biomolecule under study and the type of labeled moiety used to label it. If the biomolecule of interest is a protein and the labeled moiety is a non-radioactively labeled amino acid, then the method of detection typically should be sensitive enough to detect changes in mass of the labeled protein with respect to the unlabeled protein. In a preferred embodiment, mass spectrometry may be used to detect differences in mass between the labeled and unlabeled biomolecules. In one embodiment, gas chromatography mass spectrometry may be used. In an alternate embodiment, MALDI-TOF mass spectrometry may be used. In a preferred embodiment, high-resolution tandem mass spectrometry may be used.

Additional techniques may be utilized to separate the biomolecule (or the protein) of interest from other biomolecules in the CNS sample. As an example, immunoprecipitation may be used to isolate and purify the biomolecule (or protein) of interest before it is analyzed. On another embodiment, the biomolecule (or protein) of interest may be isolated or purified by affinity chromatography or immunoaffinity chromatography. Alternatively, mass spectrometers having chromatography setups may be used to isolate biomolecules (or proteins) without immunoprecipitation, and then the biomolecule (or protein) of interest may be measured directly. In an exemplary embodiment, the protein of interest may be immunoprecipitated and then analyzed by a liquid chromatography system interfaced with a tandem MS unit equipped with an electrospray ionization source (LC-ESI-tandem MS).

The invention also provides that multiple biomolecules in the same CNS sample may be measured simultaneously. That is, both the amount of unlabeled and labeled biomolecule (or protein) may be detected and measured separately or at the same time for multiple biomolecules (or proteins). As such, the invention provides a useful method for screening changes in production and clearance of biomolecules (or proteins) on a large scale (i.e. proteomics/metabolomics) and provides a sensitive means to detect and measure biomolecules (or proteins) involved in the underlying pathophysiology. Alternatively, the invention also provides a means to measure multiple types of biomolecules. In this context, for example, a protein and a lipid may be measured simultaneously or sequentially.

### (g) Metabolism Analysis

Once the amount of labeled and unlabeled biomolecule has been detected in a CNS sample, the ratio or percent of labeled biomolecule may be determined. If the biomolecule of interest is a protein and the amount of labeled and unlabeled protein has been measured in a CNS sample, then the ratio of labeled to unlabeled protein may be calculated. Protein metabolism (production rate, clearance rate, lag time, half-life, etc.) may be calculated from the ratio of labeled to unlabeled protein over time. There are many suitable ways to calculate these parameters.

The invention allows measurement of the labeled and unlabeled protein at the same time, so that the ratio of labeled to unlabeled protein, as well as other calculations, may be made. Those of skill in the art will be familiar with the first order kinetic models of labeling that may be used with the method of the invention. For example, the fractional synthesis rate (FSR) may be calculated. The FSR equals the initial rate of increase of labeled to unlabeled protein divided by the precursor enrichment. Likewise, the fractional clearance rate (FCR) may be calculated. In addition, other parameters, such as lag time and isotopic tracer steady state, may be determined and used as measurements of the protein's metabolism and physiology. Also, modeling may be performed on the data to fit multiple compartment models to estimate transfer between compartments. Of course, the type of mathematical modeling chosen will depend on the individual protein synthetic and clearance parameters (e.g., one-pool, multiple pools, steady state, non-steady-state, compartmental modeling, etc.).

The invention provides that the production of protein is typically based upon the rate of increase of the labeled/unlabeled protein ratio over time (i.e., the slope, the exponential fit curve, or a compartmental model fit defines the rate of protein production). For these calculations, a minimum of one sample is typically required (one could estimate the baseline label), two are preferred, and multiple samples are more preferred to calculate an accurate curve of the uptake of the label into the protein (i.e., the production rate). If multiple samples are used or preferred, the samples need not be taken from the same subject. For instance, proteins may be labeled in five different subjects at time point zero, and then a single sample taken from each subject at a different time point post-labeling.

Conversely, after the administration of labeled amino acid is terminated, the rate of decrease of the ratio of labeled to unlabeled protein typically reflects the clearance rate of that protein. For these calculations, a minimum of one sample is typically required (one could estimate the baseline label), two are preferred, and multiple samples are more preferred to calculate an accurate curve of the decrease of the label from the protein over time (i.e., the clearance rate). If multiple samples are used or preferred, the samples need not be taken from the same subject. For instance, proteins may be labeled in five different subjects at time point zero, and then a single sample taken from each subject at a different time point post-labeling. The amount of labeled protein in a CNS sample at a given time reflects the production rate or the clearance rate (i.e., removal or destruction) and is usually expressed as percent per hour or the mass/time (e.g., mg/hr) of the protein in the subject.

In an exemplary embodiment, as illustrated in the examples, the in vivo metabolism of an ApoE protein (or Aβ protein) is measured by administering a bolus of labeled leucine to a subject and collecting CNS samples. The CNS sample may be collected from various CNS issues including, but not limited to, the cerebral cortex or the hippocampus. The amounts of labeled and unlabeled ApoE (or Aβ) in the CNS samples are typically determined by immunoprecipitation followed by LC-ESI-tandem MS. From these measurements, the ratio of labeled to unlabeled ApoE (or Aβ) may be determined, and this ratio permits the determination of metabolism parameters, such as rate of production and rate of clearance of ApoE (or Aβ).

### (h) Applications

The method of the invention may be used to diagnose or monitor the progression of a neurological or neurodegenerative disease by measuring the in vivo metabolism of a CNS-derived biomolecule in a subject. The metabolism of the CNS derived biomolecule may be linked to a neurological or neurodegenerative disease such that an increase in production, a decrease in production, an increase in clearance, or a decrease in clearance in one or more CNS samples may be indicative of the presence or progression of the disease.

Additionally, the method of the invention may be used to monitor the treatment of a neurological or neurodegenerative disease by measuring the in vivo metabolism of a CNS-derived biomolecule in a subject. The metabolism of the CNS derived biomolecule may be linked to the neurological or neurodegenerative disease such that an increase in production, a decrease in production, an increase in clearance, or a decrease in clearance in one or more CNS samples may be indicative of stabilization or regression of the disease.

### II. Methods for Determining Whether a Therapeutic Agent Affects the Metabolism of a Biomolecule

Another aspect of the present invention provides a method for assessing whether a therapeutic agent used to treat a neurological or neurodegenerative disease affects the metabolism of a biomolecule produced in the CNS of a subject. For example, the metabolism of the biomolecule may be measured to determine if a given therapeutic agent results in an increase in production, a decrease in production, an increase in clearance, or a decrease in clearance of the biomolecule. Accordingly, use of this method will allow those of skill in the art to accurately determine the degree of decreased production or increased clearance of the biomolecule of interest, and correlate these measurements with the clinical outcome of the disease modifying treatment. Results from this method, therefore, may help determine the optimal doses and frequency of doses of a therapeutic agent, may assist in the decision-making regarding the design of clinical trials, and may ultimately accelerate validation of effective therapeutic agents for the treatment of neurological or neurodegenerative diseases.

In particular, the method of the invention may be used to predict which subjects will respond to a particular therapeutic agent. For example, subjects with increased production of a biomolecule may respond to a particular therapeutic agent differently than subjects with decreased clearance of the biomolecule. In particular, results from the method may be used to select the appropriate treatment (e.g., an agent that blocks the production of the biomolecule or an agent that increases the clearance of the biomolecule) for a particular subject. Similarly, results from the method may be used to select the appropriate treatment for a subject having a particular genotype (e.g., ApoE4 vs. ApoE3).

The method comprises administering a therapeutic agent and a labeled moiety to the subject, wherein the labeled moiety is incorporated into the biomolecule as it is produced in the CNS. In one embodiment, the therapeutic agent may be administered to the subject prior to the administration of the labeled moiety. In another embodiment, the labeled moiety may be administered to the subject prior to the administration of the therapeutic agent. The period of time between the administration of each may be several minutes, an hour, several hours, or many hours. In still another embodiment, the therapeutic agent and the labeled moiety may be administered simultaneously. The method further comprises collecting at least one biological sample comprising labeled and unlabeled biomolecules, detecting the amount of labeled and unlabeled biomolecule to determine the metabolism of the biomolecule, and comparing the metabolism of the biomolecule to a control value to determine whether the therapeutic agent alters the rate of production or the rate of clearance of the biomolecule in the CNS of the subject.

Non-limiting examples of neurodegenerative diseases, biomolecules, labeled moieties, routes of administration of the labeled moiety, means of detection, and means of analysis are detailed above in sections (I)(a), (I)(b), (I)(c), (I)(d), (I)(f), and (I)(g), respectively.

### (a) Therapeutic Agents

Those of skill in the art will appreciate that the therapeutic agent can and will vary depending upon the neurological or neurodegenerative disease or disorder to be treated and/or the biomolecule whose metabolism is being analyzed. In embodiments in which the biomolecule is Aβ, non-limiting examples of suitable therapeutic agents include gamma-secretase inhibitors, beta-secretase inhibitors, alpha-secretase activators, RAGE inhibitors, small molecule inhibitors of Aβ production, small molecule inhibitors of Aβ polymerization, platinum-based inhibitors of Aβ production, platinum-based inhibitors of polymerization, agents that interfere with metal-protein interactions, proteins (such as, e.g., low-density lipoprotein receptor-related protein (LRP) or soluble LRP) that bind soluble Aβ, and antibodies that clear soluble Aβ and/or break down deposited Aβ. Other suitable AD therapeutic agents include cholesterylester transfer protein (CETP) inhibitors, metalloprotease inhibitors, cholinesterase inhibitors, NMDA receptor antagonists, hormones, neuroprotective agents, and cell death inhibitors. Many of the above mentioned therapeutic agents may also affect the in vivo metabolism of other proteins implicated in neurodegenerative disorders. Additional therapeutic agents that may affect the metabolism of tau, for example, include tau kinase inhibitors, tau aggregation inhibitors, cathepsin D inhibitors, etc. Furthermore, therapeutic agents that may affect the in vivo metabolism of synuclein include sirtuin 2 inhibitors, synuclein aggregation inhibitors, proteosome inhibitors, etc. Those of skill in the art appreciate that a variety of different therapeutic agents may be utilized in the method of the invention.

The therapeutic agent may be administered to the subject in accord with known methods. Typically, the therapeutic agent will be administered orally, but other routes of administration such as parenteral or topical may also be used. The amount of therapeutic agent that is administered to the subject can and will vary depending upon the type of agent, the subject, and the particular mode of administration. Those skilled in the art will appreciate that dosages may be determined with guidance from Goodman & Goldman's The Pharmacological Basis of Therapeutics, Tenth Edition (2001), Appendix II, pp. 475-493, and the Physicians' Desk Reference.

### (b) Biological Sample

The biological sample comprising labeled and unlabeled biomolecules collected for analysis can and will vary depending upon the embodiment. In some embodiments, the biological sample may be a body fluid. Suitable body fluids include, but are not limited to, cerebral spinal fluid (CSF), blood plasma, blood serum, urine, saliva, perspiration, and tears. In other embodiments, the biological sample may be a CNS tissue, as detailed above in section (I)(e). The biological sample generally will be collected using standard procedures well known to those of skill in the art.

### (c) Control Value

In general, the control value refers to the in vivo metabolism of the biomolecule of interest in the same subject prior to administration of the therapeutic agent, a different subject who is not administered the therapeutic agent, or a subject having a different genotype who is administered the therapeutic agent. Differences between the test subject and the control subject generally will reveal whether the therapeutic agent affects the rate of production of the biomolecule or the rate of clearance of the biomolecule. This information may be used to predict which subjects will respond to a particular therapeutic agent. Furthermore, this information may be used to determine the appropriate dose and timing of administration of a particular therapeutic agent.

### (d) Preferred Embodiment

In a preferred embodiment, the biomolecule may be Aβ, the labeled moiety may be ¹³C₆-leucine, the therapeutic agent may be used for the treatment of AD, the biological sample may be CSF; the labeled protein and unlabeled protein may be isolated from the sample by immunoprecipitation, and the labeled and unlabeled proteins may be detected by mass spectrometry.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Clearance rate” refers to the rate at which the biomolecule of interest is removed.

“CNS sample” refers to a biological sample derived from a CNS tissue or a CNS fluid. “CNS tissue” includes all tissues within the blood-brain barrier. Similarly, a “CNS fluid” includes all fluids within the blood-brain barrier.

“CNS derived cells” includes all cells within the blood-brain-barrier including neurons, astrocytes, microglia, choroid plexus cells, ependymal cells, other glial cells, etc.

“Fractional clearance rate” or FCR is calculated as the natural log of the ratio of labeled biomolecule over a specified period of time.

“Fractional synthesis rate” or FSR is calculated as the slope of the increasing ratio of labeled biomolecule over a specified period of time divided by the predicted steady state value of the labeled precursor.

“Isotope” refers to all forms of a given element whose nuclei have the same atomic number but have different mass numbers because they contain different numbers of neutrons. By way of a non-limiting example, ¹²C and ¹³C are both stable isotopes of carbon.

“Lag time” generally refers to the delay of time from when the biomolecule is first labeled until the labeled biomolecule is detected.

“Metabolism” refers to any combination of the production, transport, breakdown, modification, or clearance rate of a biomolecule.

“Production rate” refers to the rate at which the biomolecule of interest is produced.

“Steady state” refers to a state during which there is insignificant change in the measured parameter over a specified period of time.

In metabolic tracer studies, a “stable isotope” is a nonradioactive isotope that is less abundant than the most abundant naturally occurring isotope.

“Subject” as used herein means a living organism having a central nervous system. In particular, the subject is a mammal. Suitable subjects include research animals, companion animals, farm animals, and zoo animals.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the claims.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Measurement of Amyloid-β Metabolism In Vitro

### Rationale

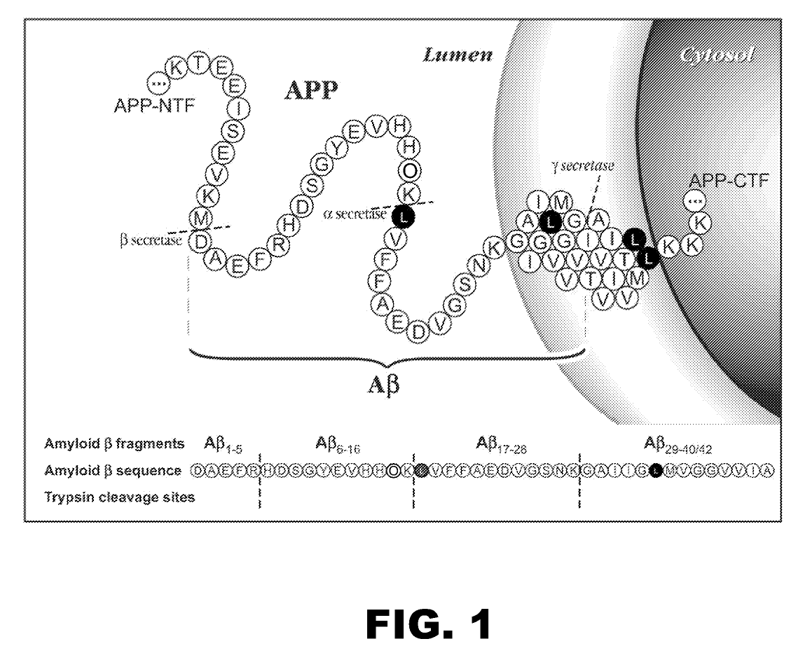

Biochemical, genetic, and animal model evidence implicates Aβ (FIG. 1) as a pathogenic peptide in AD. In order to develop a method to measure Aβ in vivo labeling, an in vitro system was designed using four basic steps: 1) label Aβ in vitro in culture, 2) isolate Aβ from other labeled proteins, 3) specifically cleave Aβ into fragments that could be analyzed for the label, and 4) quantitate the labeled and unlabeled fragments.

### Amyloid-β Immunoprecipitation and Cleavage

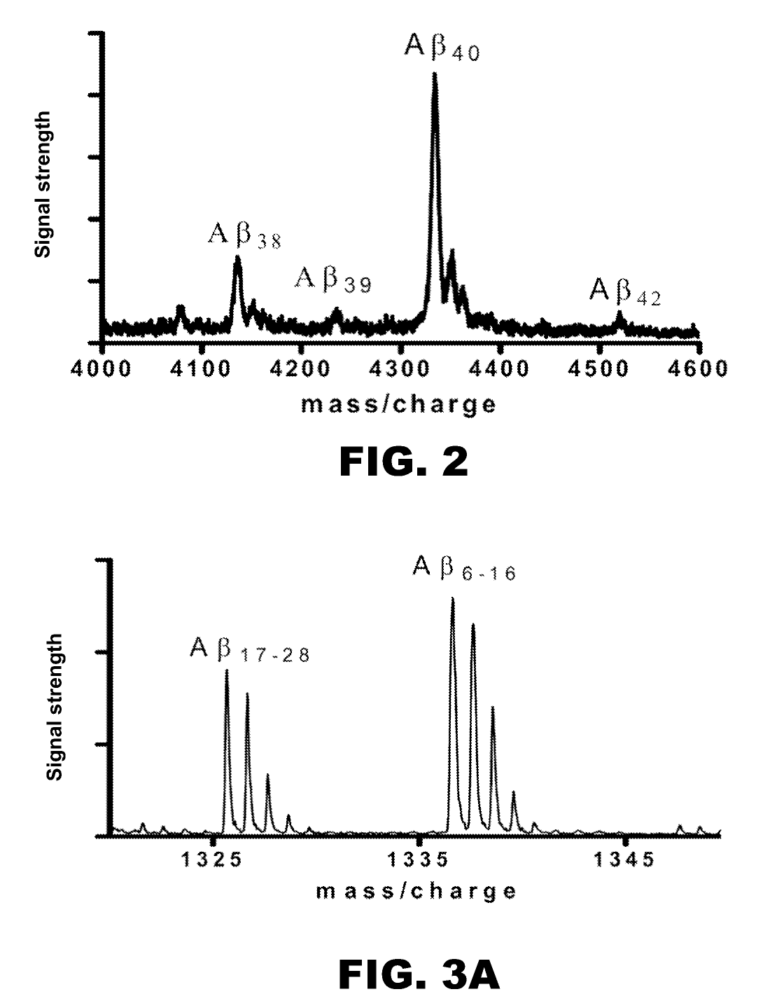

First, a method was developed for isolating and measuring unlabeled Aβ from biologic fluids. Aβ was immunoprecipitated from samples of CSF or cell culture media using a highly specific monoclonal antibody (m266), which recognizes the central domain (residues 13-28) of the molecules. Antibody beads were prepared by covalently binding m266 antibody to CNBr Sepharose beads per the manufacturers protocol at a concentration of 10 mg/ml of m266 antibody. The antibody beads were stored at 4° C. in a slurry of 50% PBS and 0.02% azide. The immunoprecipitation mixture was 250 μl of 5×RIPA, 12.5 μl of 100× protease inhibitors, and 30 μl of antibody-bead slurry in an Eppendorf tube. To this, 1 ml of the biological sample was added and the tube was rotated overnight at 4° C. The beads were rinsed once with 1×RIPA and twice with 25 mM ammonium bicarbonate. They were aspirated dry after the final rinse and Aβ was eluted off the antibody-bead complex using 30 μl of pure formic acid. Aβ was directly characterized (molecular weight and amino acid sequence) using mass spectrometry. Results were similar to previously published findings (Wang et al. 1996, J Biol. Chem. 271(50): 31894-31902), as shown in FIG. 2.

Amyloid-β may be cleaved into smaller fragments by enzymatic digestion using trypsin. Cleavage of Aβ by trypsin produces the Aβ₁₋₅, Aβ₆₋₁₆, Aβ₁₇₋₂₈, and Aβ_{29-40/42} fragments, as depicted in FIG. 1.

### Labeling of Amyloid-β

Second, a method was developed to label newly produced Aβ. ¹³C₆-leucine was used as a metabolic label because leucine equilibrates across the blood brain barrier quickly via active transport (Smith et al. 1987, J Neurochem 49(5): 1651-1658), is an essential amino acid, does not change the properties of Aβ, and is safe and nonradioactive. ¹³C stable isotopes do not change the chemical or biologic properties of amino acids or proteins; only the mass weight is increased by one Dalton for each ¹³C label. In fact, entire organisms have been grown on pure ¹³C without any deleterious effect. The labeled leucine is incorporated into the amino acid sequence of Aβ at positions 17 and 34 (see FIG. 1).

The naturally occurring isotopes ¹³C (1.1% of all carbon) and ¹⁵N cause a natural distribution of mass of larger molecules, including proteins. Due to the size of Aβ and the presence of these naturally occurring isotopes, the peptide may be broken into smaller peptides for direct measurement of the label. Alternatively, separation may be made using whole undigested Aβ.

### Liquid Chromatography/Mass Spectrometry

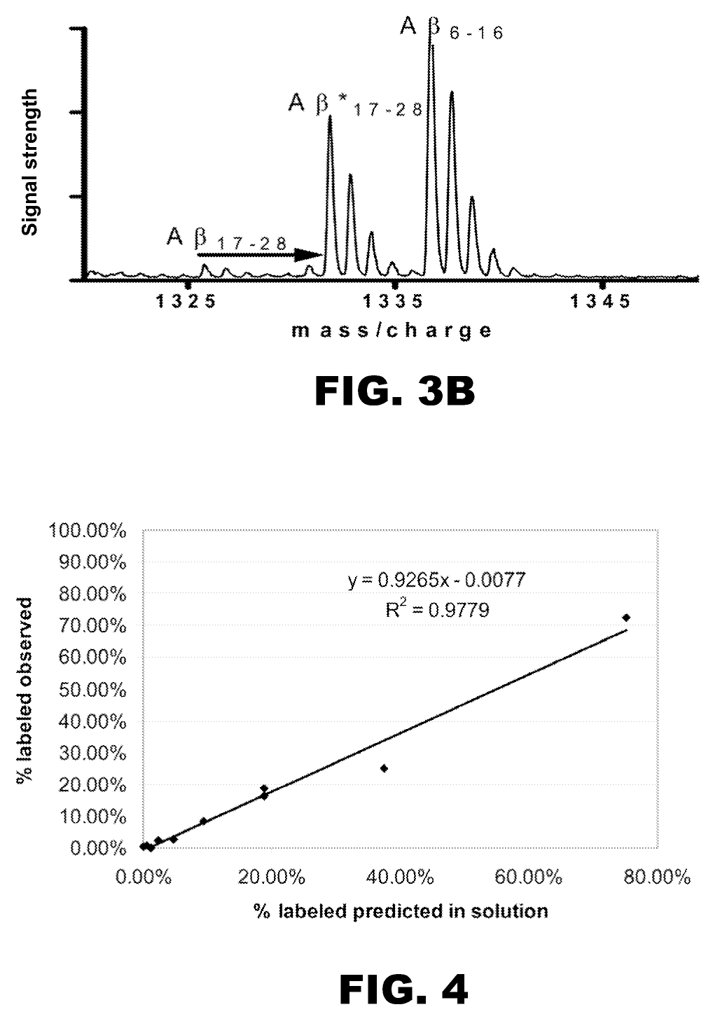

Third, a method to accurately quantitate labeled and unlabeled Aβ was developed. For this, a Waters (Milford, Mass.) capillary liquid chromatography system with auto injector was interfaced to a Thermo-Finnigan (San Jose, Calif.) LCQ-DECA equipped with an electrospray ionization source (LC-ESI-tandem MS). A 5 μl aliquot of each sample was injected onto a Vydac C-18 capillary column (0.3×150 mm MS 5 μm column). The Aβ₁₇₋₂₈ fragment contains one leucine residue and incorporation of ¹³C₆-leucine shifts the molecular weight of the fragment by 6 Daltons. In positive-ion scanning mode, LC-ESI-MS analysis of trypsin-digested synthetic and immunoprecipitated Aβ yielded the expected parent ions at masses 1325.2 for Aβ₁₇₋₂₈ and 1331.2 for ¹³C₆-leucine labeled Aβ₁₇₋₂₈ (FIGS. 3A and 3B). The percent of labeled Aβ (Aβ*) was calculated as the ratio of all labeled MS/MS ions from labeled Aβ₁₇₋₂₈ divided by all unlabeled MS/MS ions from unlabeled Aβ₁₇₋₂₈. A custom Microsoft Excel spreadsheet with macros was used to calculate the ratio as the tracer to tracee ratio (TTR) of Aβ₁₇₋₂₈ by the following formula:

$$T T {R}_{A β} = \frac{\sum MS \text{/} MS ions A {β}_{17 \text{-} 28}^{*}}{\sum MS \text{/} MS ions A {β}_{17 \text{-} 28}}$$

It was concluded that this method provided a highly specific “fingerprint” of the Aβ in both labeled and unlabeled forms, as it quantitated the amounts of each form and determined the amino acid sequence at the same time. In this way, excellent separation and specificity of the labeled to unlabeled Aβ peptide was achieved. Accuracy and precision were tested by generating a standard curve from serial dilutions of labeled and unlabeled culture media (FIG. 4). The linear fit from a range of 0% to 80% labeled Aβ serial dilution standard curve gave an R² of 0.98 and slope of 0.92. Alternative measuring techniques that were evaluated included measuring parent ions directly in selective ion mode only and also using a MALDI-TOF mass spectrometer. However, these methods were unable to offer the sensitivity and specificity that was achieved by the LC-ESI using quantitative tandem mass spectrum analysis.

### Amyloid-β In Vitro Labeling

Human neuroglioma cells that produce Aβ (Murphy et al. 2000, J Biol. Chem. 275(34): 26277-26284) were grown in the presence of ¹³C₆-labeled leucine (Cambridge Isotope Laboratories, Cambridge, Mass.) or unlabeled leucine. Aβ was isolated from the media by immunoprecipitation with m266 antibody (see above). The eluted Aβ was digested with trypsin for 4 hours at 37° C., and the fragments were analyzed by LC-ESI MS. As expected, the Aβ₁₇₋₂₈ fragment of Aβ isolated from cells incubated in the presence of unlabeled leucine had a molecular weight of 1325.2 and the Aβ₁₇₋₂₈ fragment of Aβ isolated from ¹³C₆-labeled leucine incubated cells had a molecular weight of 1331.2 (FIG. 3B). These findings indicate that the cells incorporated ¹³C₆-leucine into Aβ, confirming that Aβ produced in the presence of ¹³C₆-leucine incorporates the labeled amino acid and that the shift of 6 Daltons in the molecular weight of the leucine-containing peptide can be distinguished using mass spectrometry.

Cell culture media at 4 hours and 24 hours of ¹³C₆-leucine labeling were analyzed to determine the relative amount of labeling that occurs as a function of time. The 4-hour labeling experiment revealed approximately 70% labeling, while the 24-hour labeling experiment revealed more than 95% labeling. These findings indicate that within hours after exposure to the label, amyloid precursor protein (APP) incorporated the labeled amino acid, and the labeled Aβ was cleaved from labeled APP and released into the extracellular space.

### Example 2

### Measurement of Amyloid-β Metabolism In Vivo

### Rationale

Protein production and clearance are important parameters that are tightly regulated and reflect normal physiology as well as disease states. Previous studies of protein metabolism in humans have focused on whole body or peripheral body proteins, but not on proteins produced in the central nervous system (CNS). No methods were previously available to quantify protein production or clearance rates in the CNS of humans. Such a method would be valuable to assess not only Aβ production or clearance rates in humans but also the metabolism of a variety other proteins relevant to diseases of the CNS. In order to address critical questions about underlying AD pathogenesis and Aβ metabolism, a method for quantifying Aβ fractional synthesis rate (FSR) and fractional clearance rate (FCR) in vivo in the CNS of humans was developed.

### Labeled Leucine Quantitation

Plasma and CSF samples were analyzed to determine the amount of labeled leucine present in each fluid. The labeled to unlabeled leucine ratios for plasma and CSF ¹³C₆-leucine were quantified using capillary gas chromatography-mass spectrometry (GC-MS) (Yarasheski et al. 2005, Am J Physiol. Endocrinol. Metab. 288: E278-284; Yarasheski et al. 1998 Am J Physiol. 275: E577-583), which is more appropriate than LC-ESI-MS for low mass amino acid analysis. The ¹³C₆-leucine reached steady state levels of 14% and 10% in both plasma and CSF, respectively, within an hour. This confirmed that leucine was rapidly transported across the blood-brain-barrier via known neutral amino acid transporter systems (Smith et al. 1987 J Neurochem. 49(5): 1651-1658).

### Labeled Aβ Dynamics

For each sample of CSF collected, the ratio of labeled to unlabeled Aβ was determined by immunoprecipitation-MS/MS, as described above. The MS/MS ions from ¹³C₆-labeled Aβ₁₇₋₂₈ were divided by the MS/MS ions from unlabeled Aβ₁₇₋₂₈ to produce a ratio of labeled Aβ to unlabeled Aβ (see TTR formula, above). There was no measurable labeled Aβ for the first 4 hours, followed by an increase from 5 to 13 hours. There was no significant change from 13 to 24 hours. The labeled Aβ decreased from 24 to 36 hours.

### Calculation of FSR and FCR:

The fractional synthesis rate (FSR) was calculated using the standard formula, presented below:

$$F S R = \frac{{( {E}_{t 2} - {E}_{t 1} )}_{A β}}{( {t}_{2} - {t}_{1} )} \div Precursor E$$

Where (Eₜ₂−Eₜ₁)_{Aβ}/(t₂−t₁) is defined as the slope of labeled Aβ during labeling and the Precursor E is the ratio of labeled leucine. FSR, in percent per hour, was operationally defined as the slope of the linear regression from 6 to 15 hours divided by the average of CSF ¹³C₆-labeled leucine level during infusion. For example, a FSR of 7.6% per hour means that 7.6% of total Aβ was produced each hour.

The fractional clearance rate (FCR) was calculated by fitting the slope of the natural logarithm of the clearance portion of the labeled Aβ curve, according to the following formula:

$$F C R = ln ( \frac{Δ T T {R}_{A β}}{Δ time {( hours )}_{24 \text{-} 36}} )$$

The FCR was operationally defined as the natural log of the labeled Aβ from 24 to 36 hours. For example, a FCR of 8.3% per hour means that 8.3% of total Aβ was cleared each hour. The average FSR of Aβ for these 6 healthy young participants was 7.6%/hr and the average FCR was 8.3%/hr. These values were not statistically different from each other.

### Example 3

### Determination of the Effect of ApoE Genotype on CSF Aβ Metabolism

### Rationale

ApoE genotype is a well-validated genetic risk factor for AD. Immunohistochemical studies revealed that ApoE co-localized to extracellular amyloid deposits in AD. Furthermore, ApoE ε4 genotype was found to be a risk factor for AD in human populations. The ApoE ε2 allele has been shown to be protective in the risk of AD. ApoE genotype has also been shown to dramatically effect changes in AD pathology in several mouse models of AD (Holtzman et al. 2000 PNAS 97(2892); Fagan et al. 2002 Neurobiol. Dis 9 (305); Fryer et al. 2005 J Neurosci 25 (2803))

ApoE ε4 dose dependently increases the density of Aβ deposits in AD and in cerebral amyloid angiopathy (CAA). ApoE is associated with soluble Aβ in CSF, plasma and in normal and AD brain. It is likely that ApoE4 is associated with AD and CAA through the common mechanism of influencing Aβ metabolism, although ApoE4 has been shown to be involved in a variety of other pathways.

ApoE isoform has been shown to cause dose and allele dependent changes in time of onset of Aβ deposition and distribution of Aβ deposition in mouse models of AD (Holtzman et al., 2000, Proc. Natl. Acad. Sci. 97: 2892-2897; DeMattos et al. 2004, Neuron 41(2): 193-202). Human ApoE3 was shown to cause a dose dependent decrease in Aβ deposition. In addition, clearance studies have shown that Aβ t_{1/2} in brain interstitial fluid is ˜1-2 hours, which is decreased without ApoE. Together, this suggests that ApoE has Aβ binding and clearance effects on CNS Aβ.

### Experimental Design and Analysis

ApoE genotype can be determined in each participant. The Buffy coat (white blood cell layer) from centrifuged plasma can be collected and immediately frozen at −80° C. using standard techniques known to those of skill in the art. The ApoE genotype of the sample is determined by PCR analysis (Talbot et al. 1994, Lancet 343(8910): 1432-1433). The effect of gene dose of ApoE2 (0, 1, or 2 copies) and ApoE4 (0, 1, or 2 copies) can be analyzed with the continuous variable of CSF or plasma FSR or FCR of Aβ metabolism.

Methods for statistical analysis can be made using standard techniques known to those of skill in the art. For example, for the FSR and FCR of Aβ, a two-way or three-way ANOVA can be performed with human ApoE isoform and age as factors in the control group and also in the AD group. If the data are not normally distributed, a transformation can be utilized to meet necessary statistical assumptions regarding Gaussian distributions.

### Results

It is expected that ApoE4 can decrease clearance of Aβ compared to ApoE3 in the CNS. Conversely, ApoE2 is expected to increase clearance of Aβ compared to ApoE3 in the CNS. A change in production rate of Aβ based on ApoE genotype is not expected. If changes in Aβ metabolism are detected, this would be evidence of the effect of ApoE status on in vivo Aβ metabolism in humans.

### Example 4

### In Vivo Stable Isotope-Labeling and ApoE Immunoprecipitation-Mass Spectroscopy

Human ApoE2 and ApoE4 knock-in mice were intraperitoneally injected with ¹³C₆-labeled leucine (Cambridge Isotope Laboratories, Cat. # CLM-2262-0.1) at 200 mg/kg dose and then their brain tissues (cortex and hippocampus) were harvested and lysed in 1% Triton-X 100 lysis buffer (Roche, Cat. # 11-332-481-001) at pre-determined time points. ApoE from the brain tissue lysate was immunoprecipitated with anti-ApoE antibody (WUE4) coupled to Protein G Sepharose beads (GE Healthcare, Cat. # 17-0618-014). Immunoprecipitated ApoE was eluted using 100% formic acid (Sigma, Cat. # 94318-50ML-F). The purified ApoE was then digested with sequencing grade modified trypsin (Promega, Cat. # V5111). After tryptic digestion, the supernatant containing ApoE peptides was analyzed by nano-LC tandem MS (Thermo-Finnigan LTQ equipped with a New Objective nanoflow ESI source) as previously described (Bateman et al., 2006, Nature Medicine, 12:856-861). Percent labeled ApoE was calculated from the ratio of the labeled to unlabeled product ions for ApoE tryptic peptide SWFEPLVEDMQR (SEQ ID NO:2). The fractional clearance rate (FCR) was calculated by using the slope of the natural log of percent labeled ApoE.

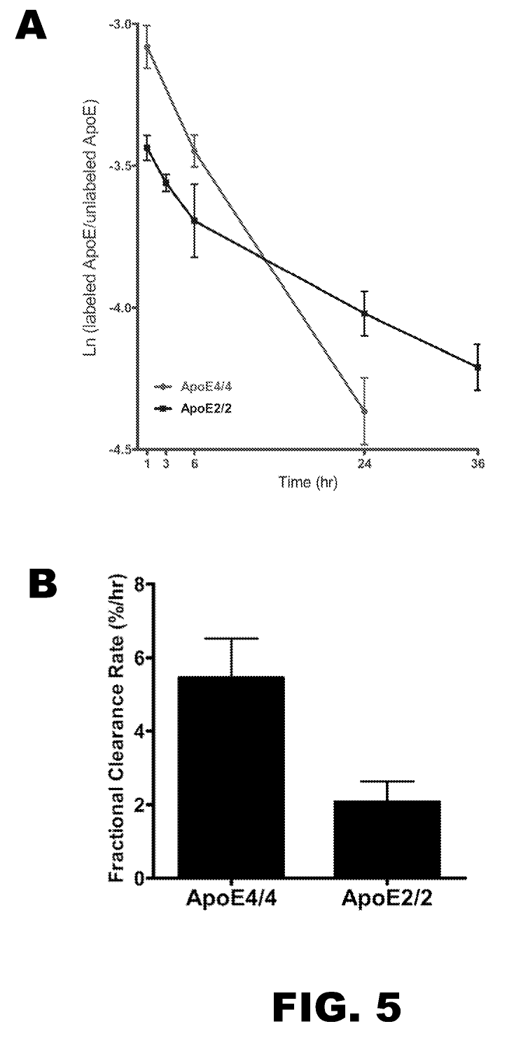

Panel A of FIG. 5 illustrates the fraction of labeled ApoE to unlabelled ApoE at 1, 3, 6, 24, and 36 hours after labeling. Panel B shows the fractional clearance rate of ApoE4 in ApoE4/4 knock-in mice in the cortex (5.456%/h5; 95% confidence intervals: 4.394-6.518%) and ApoE2 in ApoE2/E2 knock-in mice in the cortex (2.072%/hr; 95% confidence intervals: 1.506-2.638%/hr). The ApoE4/4 subjects were 10-12 months old and the ApoE2 subjects were 2-3 months old.

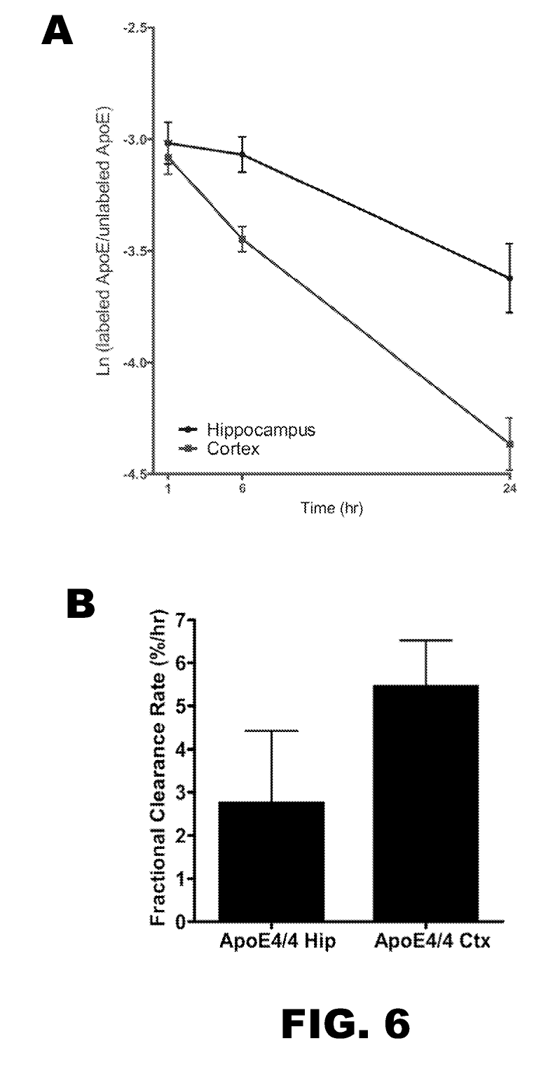

Panel A of FIG. 6 illustrates the fraction of labeled ApoE to unlabelled ApoE at 1, 6, and 24 hours after labeling. Panel B shows the fractional clearance rate of ApoE4 in the hippocampus (hip) of ApoE4/E4 knock-in mice (2.749%/hr; 95% confidence intervals: 1.074-4.423%) and ApoE4 in the cortex of ApoE4/E4 knock-in mice (5.456%/hr; 95% confidence intervals: 4.394-6.518%/hr).

### Example 5

### Amyloid-β Production and Clearance in Mouse Brain

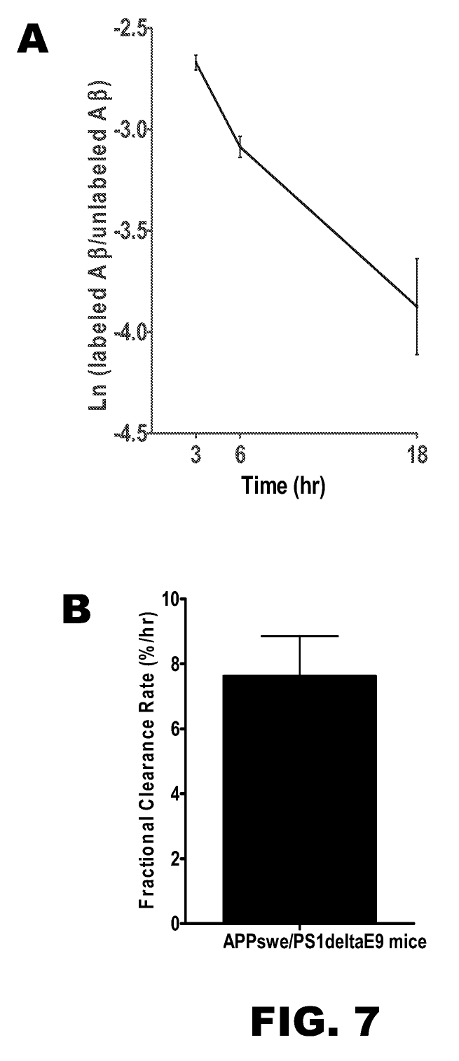

To measure the brain Aβ clearance rate in mice, the method developed to assess ApoE clearance described above in Example 4 was performed in APPswe/PS1deltaE9 double transgenic mice (line #85; Jackson Labs, Bar Harbor, Me.). After a bolus intraperitioneal injection of ¹³C₆-leucine (200 μg/g), cortical brain tissue was harvested at 3, 6, and 18 hr post-injection (n=3) and lysed (lysis buffer, 1% Triton with PBS). Aβ was immunoprecipitated with an Aβ monoclonal antibody (HJ5.2, directed to Aβ 13-28) and then analyzed by nano-LC-MS/MS as described above. FIG. 7A shows the fraction of labeled Aβ to unlabelled Aβ at 3, 6, and 18 hours after labeling. FIG. 7B presents the fractional clearance rate (FCR) of soluble cortical Aβ (7.62±1.25% per hour). The clearance rate of soluble Aβ from cortex of transgenic mice was very similar with that of Aβ from human CSF as described by Bateman et al. 2006, supra.

What is claimed is:

1. A method for measuring the in vivo metabolism of one or more biomolecules produced in the central nervous system in a subject, the method comprising:
(a) administering a labeled moiety to the subject, the labeled moiety being capable of crossing the blood brain barrier and incorporating into the biomolecule(s) as the one or more biomolecules are produced in the subject;
(b) obtaining a central nervous system sample from the subject, the central nervous system sample being a central nervous system tissue, the central nervous system sample comprising a labeled biomolecule fraction in which the labeled moiety is incorporated into the one or more biomolecules and an unlabeled biomolecule fraction in which the labeled moiety is not incorporated into the one or more biomolecules; and
(c) detecting the amount labeled biomolecule and the amount of unlabeled biomolecule for each of the one or more labeled biomolecules, wherein the ratio of labeled biomolecule to unlabeled biomolecule for each biomolecule is directly proportional to the metabolism of said biomolecule in the subject.

2. The method of claim 1, wherein the one or more biomolecules is selected from the group consisting of protein, lipid, nucleic acid, carbohydrate, and combinations thereof.

3. The method of claim 2, wherein the one or more proteins is selected from the group consisting of amyloid-beta, apolipoprotein E, apolipoprotein J, synuclein, soluble amyloid precursor protein, Tau, alpha-2 macroglobulin, S100B, myelin basic protein, TDP-43, huntingtin, progranulin, an interleukin, TNF, and combinations thereof.

4. The method of claim 1, wherein the labeled moiety comprises a non-radioactive isotope selected from the group consisting of ²H, ¹³C, ¹⁵N, ¹⁷O, ¹⁸O, ³³S, ³⁴S, and ³⁶S.

5. The method of claim 1, wherein the labeled moiety is an amino acid comprising ¹³C.

6. The method of claim 1, further comprising separating the one or more labeled biomolecules and the one or more unlabeled biomolecules from the central nervous system sample.

7. The method of claim 6, wherein, for each of the one or more biomolecules, the labeled biomolecule and the unlabeled biomolecule are separated from the sample by immunoprecipitation.

8. The method of claim 7, wherein the amount of labeled biomolecule and the amount of unlabeled biomolecule are detected by mass spectrometry.

9. The method of claim 1, further comprising simultaneously obtaining more than one type of central nervous system sample.

10. The method of claim 1, wherein the biomolecule is a protein selected from the group consisting of amyloid-beta and apolipoprotein E; the labeled moiety is ¹³C₆-leucine; the labeled protein and unlabeled protein are separated from the central nervous system sample by immunoprecipitation; and the labeled and unlabeled proteins are detected by mass spectrometry.

11. A method for determining whether a therapeutic agent affects the in vivo metabolism of a biomolecule produced in the central nervous system of a subject, the method comprising:
(a) administering the therapeutic agent to the subject;
(b) administering a labeled moiety to the subject, the labeled moiety being capable of crossing the blood brain barrier and incorporating into the biomolecule as the biomolecule is produced in the subject;
(c) obtaining a biological sample from the subject, the biological sample comprising a labeled biomolecule fraction in which the labeled moiety is incorporated into the biomolecule and an unlabeled biomolecule fraction in which the labeled moiety is not incorporated into the biomolecule;
(d) detecting the amount of labeled biomolecule and the amount of unlabeled biomolecule, wherein the ratio of labeled biomolecule to unlabeled biomolecule is directly proportional to the metabolism of the biomolecule in the subject; and
(e) comparing the metabolism of the biomolecule in the subject to a suitable control value, such that a change from the control value indicates the therapeutic agent affects the metabolism of the biomolecule in the central nervous system of the subject.

12. The method of claim 11, wherein step (b) is performed before step (a).

13. The method of claim 11, wherein the biomolecule is selected from the group consisting of a protein, a lipid, a nucleic acid, and a carbohydrate.

14. The method of claim 12, wherein the protein is selected from the group consisting of amyloid-beta, apolipoprotein E, apolipoprotein J, synuclein, soluble amyloid precursor protein, Tau, alpha-2 macroglobulin, S100B, myelin basic protein, TDP-43, huntingtin, progranulin, an interleukin, and TNF.

15. The method of claim 11, wherein the labeled moiety comprises a non-radioactive isotope selected from the group consisting of ²H, ¹³C, ¹⁵N, ¹⁷O, ¹⁸O, ³³S, ³⁴S, and ³⁶S.

16. The method of claim 11, wherein the labeled moiety is an amino acid comprising ¹³C.

17. The method of claim 11, wherein the biological sample is selected from the group consisting of cerebral spinal fluid, blood, urine, saliva, tears, brain tissue, and spinal cord tissue.

18. The method of claim 11, wherein the suitable control value is selected from the group consisting of the same subject prior to administration of the therapeutic agent, a control subject who is not administered the therapeutic agent, and a subject having a different genotype who is administered the therapeutic agent.

19. The method of claim 11, further comprising separating the labeled biomolecule fraction and the unlabeled biomolecule fraction from the biological sample by immunoprecipitation.

20. The method of claim 19, wherein the amount of labeled biomolecule and the amount of unlabeled biomolecule are detected by mass spectrometry.
